(19) United States
(12) Patent Application Publication
(10) Pub. No.: US 2006/0074722 A1
(43) Pub. Date: Apr. 6, 2006

(54) Computerized method and system for providing medical service and related measurement apparatus

(76) Inventor: Yu-Chung Chu, Taichung City (TW)

Correspondence Address: LADAS & PARRY, 26 WEST 61ST STREET, NEW YORK, NY, 10023, US

(21) Appl. No.: 11/234,435

(22) Filed: Sep. 23, 2005

(30) Foreign Application Priority Data
Oct. 4, 2004 (TW) 093130010

(51) Int. Cl. A61B 5/00

(52) U.S. Cl. 705/3

(57) ABSTRACT

A method and system for providing medical service. A measured data of a patient is transmitted to a database; the patient selects a physician through a selection interface; and the measured data is retrieved from the database by the selected physician so that the physician can perform a diagnosis based on the measured data.

### BACKGROUND OF THE INVENTION

1. Field of Invention

The present invention relates to a system and method for providing medical services, and particularly to a computerized system and method for providing medical services.

2. Description of Related Art

Since the ancient times, medical activities have been the important part of human life. To ensure the quality of medical service, physicians usually need be trained and pass certain examinations before they obtain the licenses to provide medical services. Under such strict standards, the number of physicians in any country is not enough. Meanwhile, physicians usually have heavy workload and it is difficult for physicians to find sufficient time to improve their knowledge or develop more advanced medical techniques.

Also, people demand higher medical quality as their daily lives improves. Still, most patients need to go to the hospitals to see the physicians. Since there are not enough physicians, it is very common that patients need to wait for a long time before they meet physicians. Usually, some measurements are indicated after a short interview with physicians and patients line up again to take measurements, e.g. X-ray, blood or urine testing. When measurement result is obtained, patients again line up to wait for physicians to make diagnoses or to receive treatment/to obtain medication based on the measured result.

Such problems are more serious for people who live far away from hospitals. There are some medical centers providing distant medical services for patients. That is, videoconference between a patient and a physician is conducted for the physician to make a diagnosis. It saves time of the patient to go to the medical center, but a lot of waiting time is still necessary under current schema for providing medical service. In fact, the procedure of seeing a physician does not change substantively and the problems addressed above still exist.

There is also a problem for patients today to seek a physician satisfying requirements of patients under current schema of medical service providing. The medical history files of patients are locked in file rooms or databases in hospitals and difficult or at least inconvenient for other physicians to use these data when patients seek help from them.

Therefore, it is beneficial to provide an improved medical service providing schema that makes patients, physicians and medical centers all win.

### SUMMARY OF THE INVENTION

In an embodiment according to the present invention, a method for providing medical services includes following steps: receiving measured data of a patient and storing the measured data in a database; assigning a physician based on a selection made by the patient via a selection interface; providing the measured data to the assigned physician for performing a diagnosis based on the measured data; and transmitting the diagnosis to the patient asynchronously or synchronously.

In another embodiment according to the present invention, a plurality of medical centers are registered in a central platform, each medical center is associated with multiple physicians. A patient selects a medical center via the central platform or selects a physician directly via the central platform. In other words, there are various choices for the patient to select medical service compared to the prior art. The central platform also deals with charging affairs between medical centers and physicians so that physicians can concentrate on their professional work. In addition, multiple-tier medical service is applicable by using the central platform. Since the registration is a logic modification to records in the database, it is convenient and easy for a physician to register in a plurality is of medical centers. It is also convenient and easy for a plurality of physicians to organize a new medical center so that fee and cost of expensive medical equipments can be shared. All work can be done by following the rules recorded in the database. Moreover, if a patient assigns a physician because of the introduction of another physician or a medical center, the introducing physician or medical center is allocated a portion of a commission amount based on a predetermined ratio recorded in the database. Besides, the platform can further be coupled to an electronic commerce platform that provides goods and/or services. If a patient buys some goods and/or services from the electronic commerce platform because the introduction of the assigned physician or the medical center, the assigned physician or the medical center is allocated a portion of a commission amount according to a predetermined ratio recorded in the database.

In yet another embodiment according to the present invention, a system for providing medical service comprises a database, a selection interface, a diagnostic interface and a transmission interface. A patient assigns a physician via the selection interface and transmits measured data to the database via the transmission interface. The assigned physician makes a diagnosis based on the measured data received from the database and transmits the diagnosis to the patient via the transmission interface.

In yet another embodiment according to the present invention, a measurement apparatus includes a controller, a measurement device, an authentication interface and a transmission interface. The measurement device is used for obtaining measured data of the patient after the authentication interface authenticates the identity of the patient. Then, the measured data are transmitted to a database via the transmission interface. In another embodiment according to the present invention, the measured data is stored in a portable storage device and brought to a medical center or a hospital by the patient for uploading the measured data. The controller controls operation between the elements of the measurement apparatus.

### BRIEF DESCRIPTION OF THE DRAWINGS

The accompanying drawings are included to provide further understanding of the invention, and constitute a part of this specification. The drawings illustrate the embodiments of the invention and, together with the description, serve to explain the principles of the invention. In the drawings,

FIG. 1 illustrates a schematic diagram for providing medical service of an embodiment according to the present invention;

FIG. 2 illustrates an operation flowchart of FIG. 1;

FIG. 3 illustrates logic relationship between a central platform and medical centers;

FIG. 4 illustrates logic relationship between medical centers, the central platform and patients;

FIG. 5 illustrates a flowchart of an embodiment according to the present invention;

FIG. 6 illustrates a schematic diagram of the measurement apparatus of an embodiment according to the present invention;

FIG. 7 illustrates a schematic diagram of the central platform of an embodiment according to the present invention;

FIG. 8 illustrates another aspect of FIG. 7;

FIG. 9 illustrates a flowchart for uploading measured data; and

FIG. 10 illustrates a schematic diagram for uploading measured data.

### DESCRIPTION OF PREFERRED EMBODIMENT

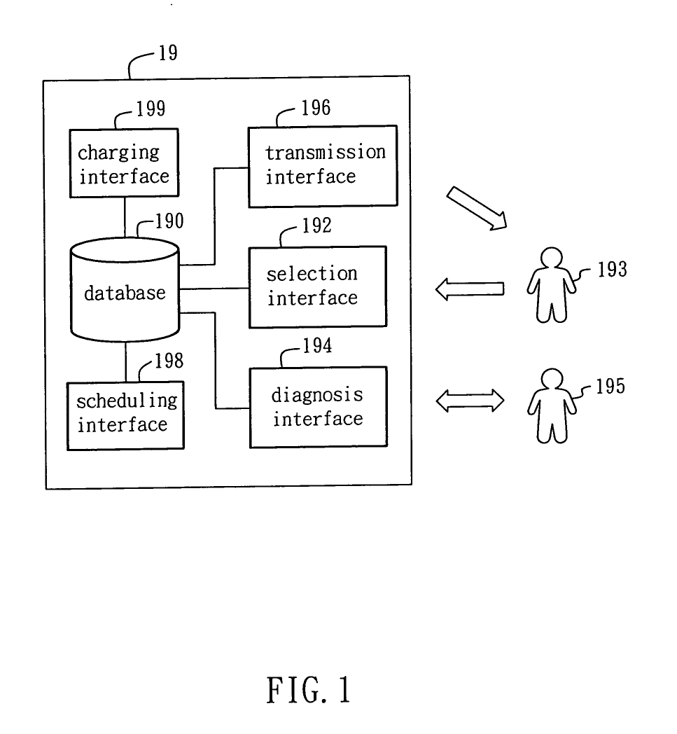

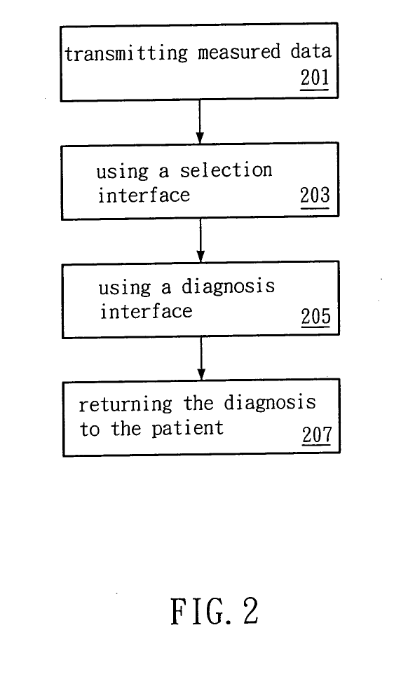

FIG. 1 illustrates a schematic diagram for providing medical service of an embodiment according to the present invention, and FIG. 2 is a flowchart illustrating the operation of the preferred embodiment. In this embodiment, a central platform 19, such as a computer system, is coupled to a network. The computer system includes a database 190, a selection interface 192, a diagnostic interface 194 and a transmission interface 196. Vital signs or other measured data like respirations, body temperature, blood pressure, heart beat, electrocardiogram, X-ray photos and blood measurements of a patient 193 are received by the database 190 of the central platform 19 and stored therein (step 201). The patient 193 assigns a physician through the selection interface 192 (step 203). The central platform 19 transmits the measured data to the assigned physician 195 via the diagnosis interface. The assigned physician 195 performs a diagnosis based on the measured data (step 205), and transmits the diagnosis to the patient 193 via the transmission interface 196 (step 207). In other words, the assigned physician 195 does not need to meet the patient 193 directly and performs a diagnosis synchronically as that in the past. Therefore, such architecture provides asynchronous feature for providing medical service. Nevertheless, synchronous medical service for the patient 193 is also applicable under such architecture and is explained in more detail below.

In implementation, it is applicable to select an open network or a closed network for data exchange between the database 190, the selection interface 192, the diagnostic interface 194 and the transmission interface 196. Examples of the open network include, but not limit to, Internet and examples of the close network include private lines. In addition, virtual private networks, or other wireless, wire, telecommunication networks or hybrid networks are options to implement the preferred embodiment. The selection interface 192, the diagnostic interface 194 and the transmission interface 196 can be implemented in software, firmware, hardware or any combination thereof. The term “interface” used here refers to any software codes, firmware codes or hardware logic circuits. More detailed explanation for implementing these interfaces is provided below.

In such system for providing medical service, a plurality of medical centers can be integrated so that the relationship between medical centers, patients and physicians are more flexible and more cost effective. For example, it only involves modification of data records in the database of the central platform. Thus, it is easy to register a physician to a plurality of medical centers, e.g. hospitals. In other aspect, it is also convenient for a plurality of physicians to organize a new medical center and share fees of medical equipments, and all these can be implemented in the database by storing fee allocation rules. Besides, if a patient assigns a physician because of the introduction of another physician or a medical center, the introducing physician or medical center is allocated a portion of a commission amount based on a predetermined ratio recorded in the database of the central platform 190. Moreover, the central platform can be coupled or integrated to another electronic commerce platform that provides a commerce transaction, e.g. selling goods and/or providing services and/or providing bid services. When one patient performs a commerce transaction via the platform because the introduction of the assigned physician or one medical center, a portion of a commission amount is allocated to the assigned physician and/or the medical center.

Rules of allocating a portion of a commission amount to medical centers and/or assigned physicians can be implemented as tables in a database. For example, in each transaction of medical service, there is a field in a table for recording whether there is an introducing (referring) medical center and/or physician. In addition, the referring commission ratio is stored in another table. Using a relational database, the two tables are operated and corresponding commission amount to the referring medical center and/or physician is obtained by programs, e.g. SQL codes. With respect to other goods and/or services transacted because of the introduction of the assigned physician and/or medical center, the commission amount can also be calculated in similar way. If the patient is the one who introduces another patient to take medical service of an assigned physician, the patient can also be allocated a portion of commission amount.

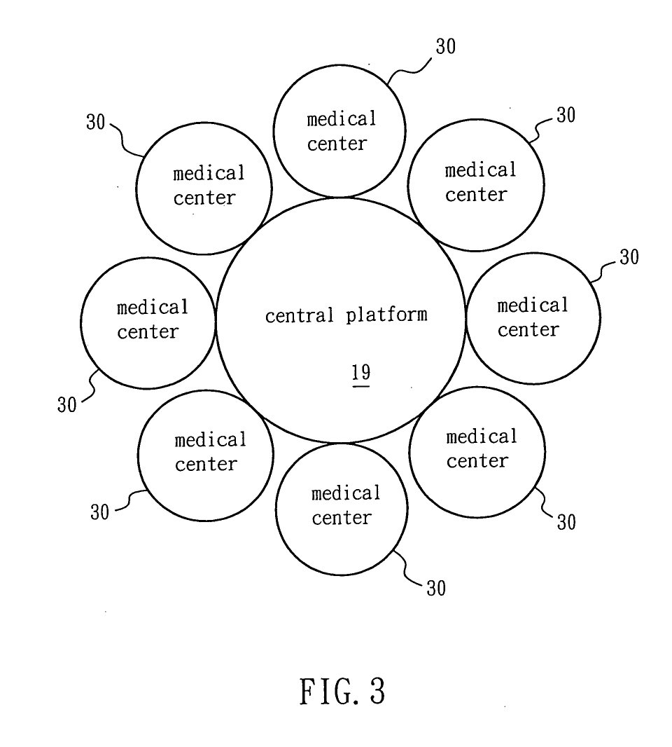

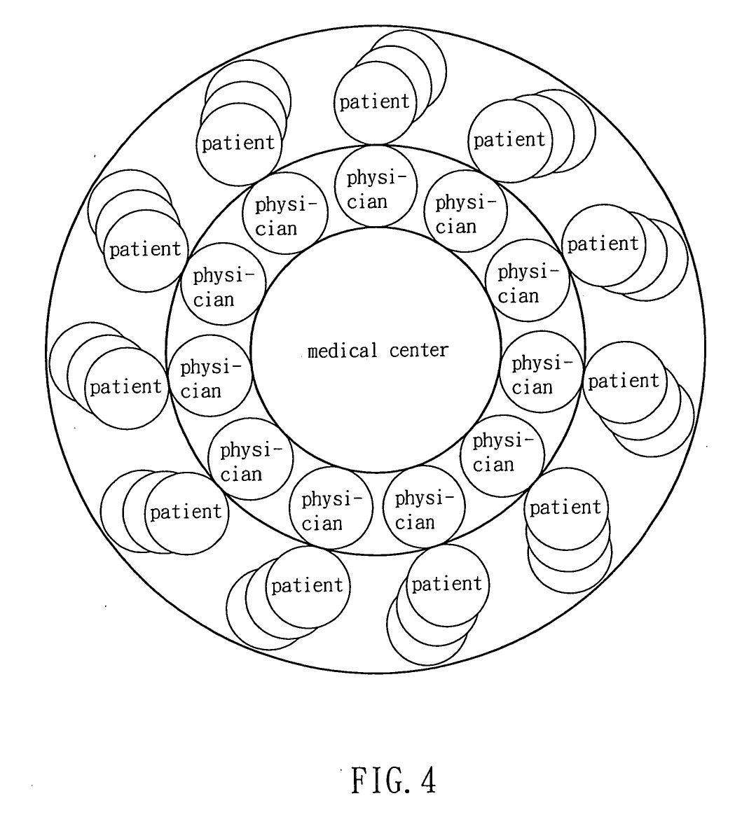

FIG. 3 illustrates that multiple medical centers 30 are registered in the central platform 19. FIG. 4 is another schematic diagram illustrating that each medical center 30 can further register a plurality of physicians 195 to be selected by a patient. Currently, medical centers, e.g. big hospitals or small clinics, provide an organization for training inexperienced physicians and sharing medical equipments. Further, many patients select a physician based on fame of an associated medical center, and such architecture has commercial applicability. Nevertheless, if data of these medical centers 30 and physicians 195 are stored in the database 190 illustrated in FIG. 1, it is very flexible to associate a physician to multiple medical centers 30.

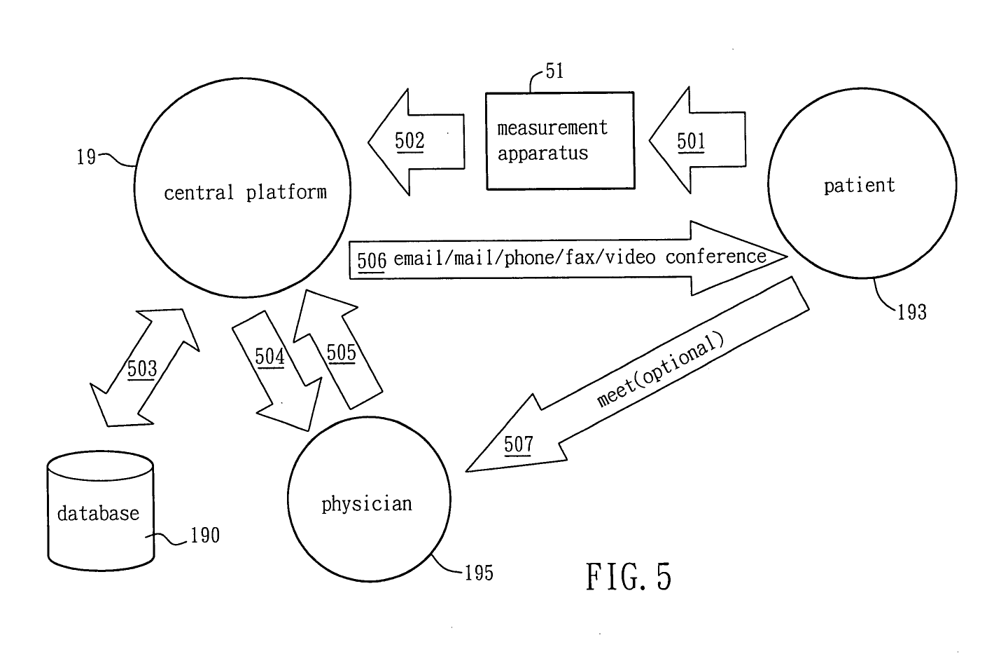

FIG. 5 is a flowchart illustrating how a patient 193 obtains medical service provided by a physician 195. The patient 193 uses a measurement apparatus 51, owned by the patient 193, a medical center 30 or a third party, to retrieve measured data of body condition. Examples of the measured data include pulse, blood pressure, breathing, body temperature, face images, throat photos, X-ray photos, test result of blood and urine, and electrocardiogram. With proper design to the measurement apparatus, e.g. a text or multimedia interaction interface, it is also applicable to collect self-description data of the patient 193 and provide the patient 193 with auxiliary information, e.g. what to be measured and/or where to take corresponding measurement.

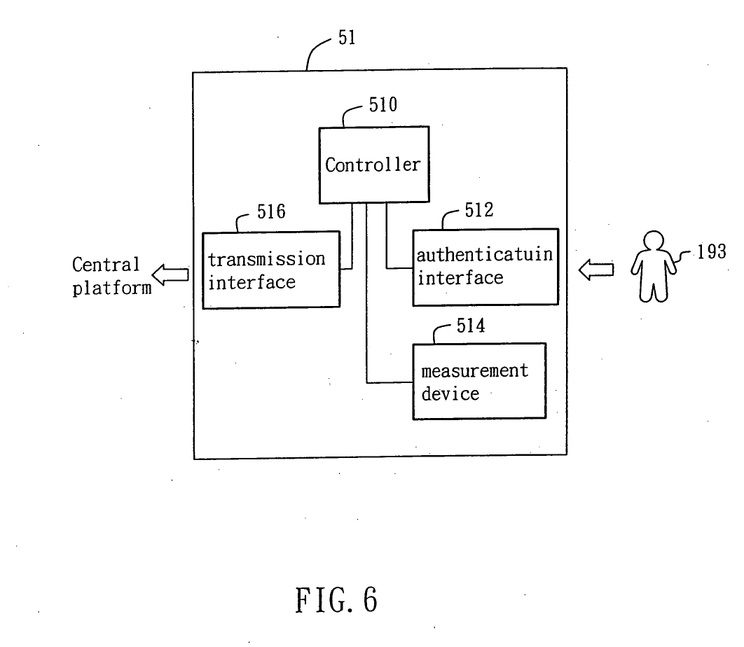

FIG. 6 illustrates a schematic diagram of the measurement apparatus of an embodiment according to the present invention. The measurement apparatus 51 has an authentication interface 512, a measurement device 514, a transmission interface 516 and a controller 510. The authentication interface 512 is used for authenticating the identity of a patient 193. The measurement device 514 is used for measuring vital signs or other corresponding information of the patient 193. The transmission interface 516 transmits the measured data as well as the identity data of the patient 193 to the central platform 19. The controller 510 is used for controlling operations among elements of the measurement apparatus 51. Such measurement apparatus 51 is particularly suitable for expensive equipment or equipments that need to operate by well-trained persons. In past, these equipments are installed in medical centers and measured data are transmitted to corresponding physicians directly via internal channel and/or network. In contrast, in the method and system for providing medical service of the present invention, a measurement apparatus 51 can be disposed in many locations, or even installed in some stores that provide 24-hour service and are shared by a plurality of medical centers and/or physicians. For the great majority of patients, it is convenient and enough for these patients to go to the site where the measurement apparatus 51 is provided to collect necessary data for preliminary diagnosis. In other words, most patients do not need to go to the hospitals via a long distance and line up there for a long time just to collect some basic measured data before they see their physicians.

Now referring back to FIG. 5, when the measured data of the patient 193 are collected, the measured data are transmitted to the central platform 19 (step 502). Another option is to store the measured data in a portable storage device that has authentication mechanism. The measured data in the portable storage device are then transmitted to the central platform 19 via the transmission interface or uploaded to the computer located in the hospital. Examples of the portable storage device that has authentication mechanism include portable disks, flash memory devices, mini hard drives, and optical disks. The central platform 19 stores the measured data in the database 190 (step 503). When the assigned physician 195 opens the diagnostic interface 194, the measured data and corresponding medical history of the patient are transmitted to the assigned physician 195 (step 504). The diagnostic interface 194 is implemented in the form of computer software, firmware or hardware and can be configured to provide preliminary analysis of the measured data and the medical history of the patient and items that need to be noticed. Such approach collects various symptoms and criteria for determining diseases. With expert systems and/or data mining, auxiliary tools and information in time ensure that the assigned physician 195 makes a correct diagnosis efficiently.

When the assigned physician 195 makes a preliminary diagnosis, the diagnosis is input to the diagnostic interface (step 505). For minor diseases or symptoms that can be recognized directly from the measured data, the diagnosis includes medicine prescription and authentication data. Further, the diagnosis may include additional information about the medicine prescription, e.g. side effect of particular medicines, taking time, and corresponding health information, generated by the central platform 17 automatically according to the diagnosis content. Such additional information can be generated automatically, without wasting precious time of the physicians, but improve the quality of medical service significantly. For those situations where further measurements need to be taken, the diagnosis may include authentication data and an instruction to take further measurement. In addition, an appointment between the patient 193 and the physician can be scheduled if direct contact is necessary. For such case, a scheduling interface 198 is provided in the central platform 19 to manually, automatically or semi-automatically schedule a proper date for the assigned physician and the patient and the scheduled date is included in the diagnosis.

The central platform 19 transmits the diagnosis to the patient 193 via an asynchronous and/or synchronous media. Examples of the asynchronous media include e-mails, mails, telephone messages. Examples of the synchronous media include telephone calls and video conferences. When the medical service is finished, the central platform provides a charging interface 199 for charging a medical fee for the medical service. The collected medical fee is then allocated to corresponding medical centers 30 and/or the assigned physicians.

To ensure reliability for providing medical service, certification authentication, e.g. encryption like SSL, PKI, is embedded in the central platform 19. Other approaches for ensuring the identity of the patient and the physician include using smart cards, finger prints or other biometrics techniques.

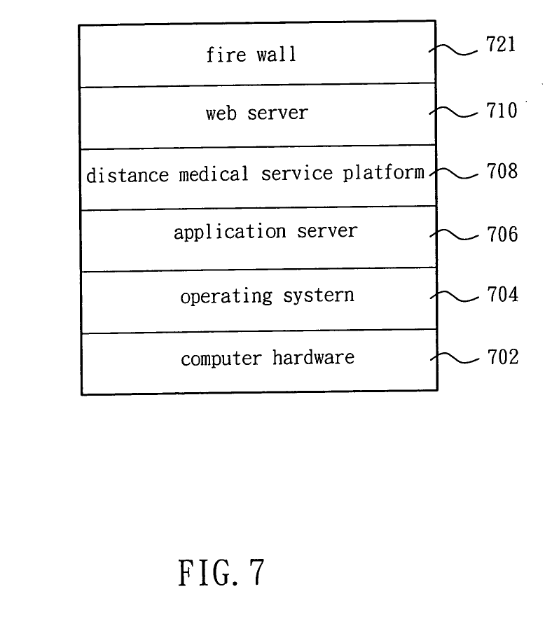

FIG. 7 illustrates an example of the central platform 19 mentioned above. In FIG. 7, the selection interface 192, the diagnostic interface 194, the transmission interface 196, the scheduling interface 198 and the charging interface 199 are implemented in software programs, including message delivery and authentication programs that construct a distance medical service platform 708. The distance medical service platform 708 can be deployed upon a web server 710 like Apache or IIS and a firewall 712. In addition, the distance medical service platform 708 may utilize an application server 706, an operating system 704 and computer hardware 702 to perform its tasks.

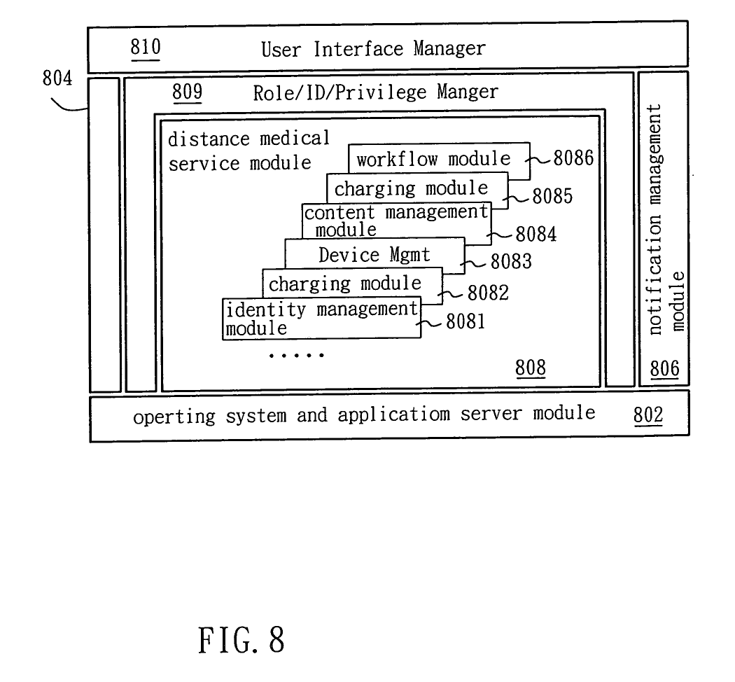

FIG. 8 is a logic diagram illustrating major components of the central platform 17 that is implemented using software approach. There is a presentation management module 804, a notification management module 806 and a distance medical service module 808. The distance medical service module 808 includes multiple modules, like identity authentication management module 8081, a charging module 8082, a device management module 8083, a content management module 8084, a charging module 8085, a workflow module 8086. These modules can be implemented in object oriented languages or other programming languages upon different framework, e.g. Java Beans, COM object architecture, UNIX programming architecture. Identity management for physicians 195, patients 193 and medical centers 30 are called identity authentication management module 809. Under such architecture, user interfaces may be designed in response to various requirements and be managed by user interface management module 810.

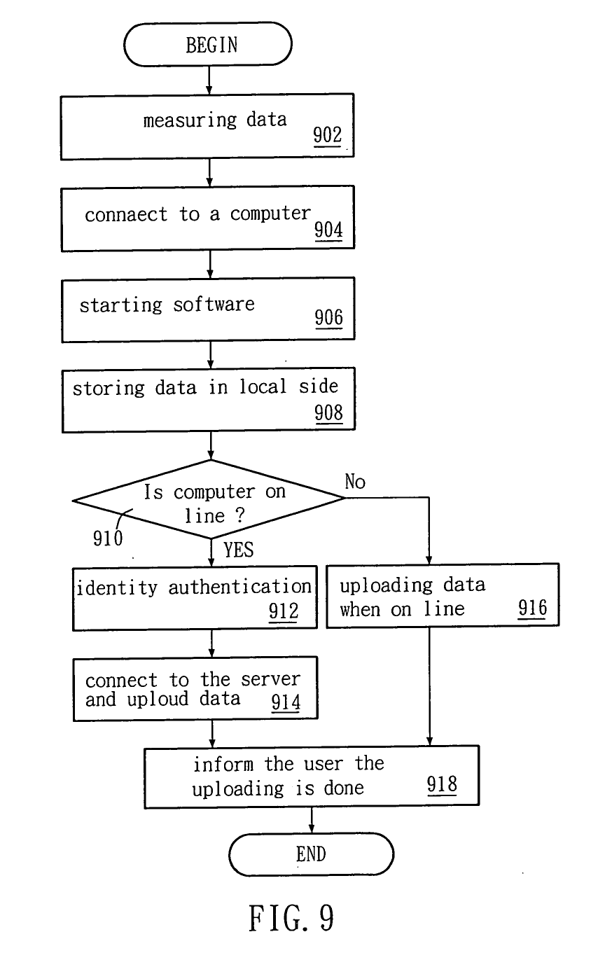

FIG. 9 is a flowchart illustrating how the patient 193 transmits the measured data from a home computer or a computer placed in a particular site to the central platform 19. First, the measured data are obtained (step 902). If the measurement apparatus is equipped with a USB or RS232 interface, the measurement apparatus is connected to the computer (step 904). The computer executes an interface program in the client site (step 906). With the interface program, the measured data are stored in the local computer first (step 908). Next, the interface program detects whether the computer is connected to a network (step 910). If the computer is currently connected to a network, the interface program connects itself to the central platform, and performs identity authentication (step 912) and an operation of uploading the measured data (step 914). If the computer is not connected to a network, the interface program sets a status that waits for uploading the measured data until the computer is connected on line (step 916). Next, the patient 193 is informed that the measured data have been uploaded (step 918).

To elder people, it is usually not a simple task to operate computers. However, with Bluetooth or other wireless technologies, home gateways that access data of peripheral devices are getting more and more popular. Therefore, the preferred embodiment is applicable in such environment.

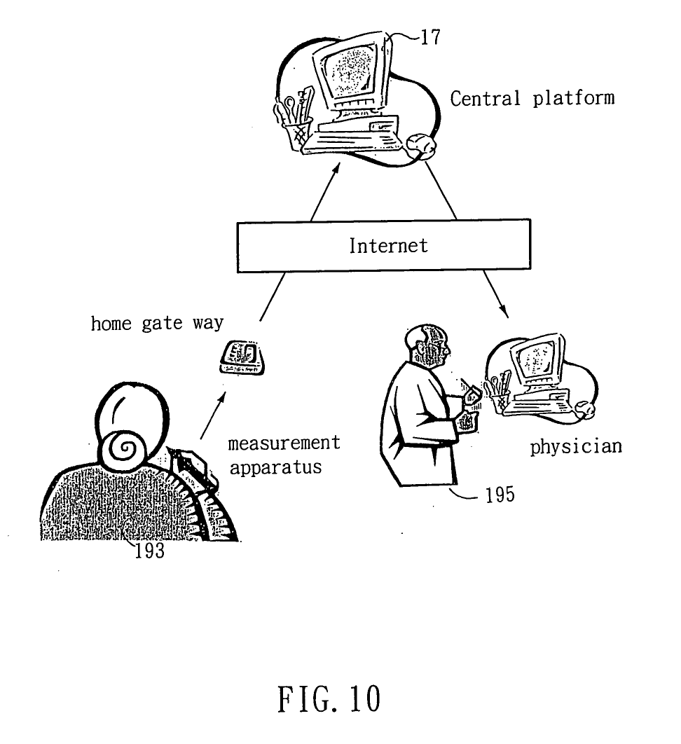

FIG. 10 illustrates an example of such application. The measured data of the patient 193 obtained by a measurement apparatus are received by a setup box or a home gateway automatically. The measured data are then transmitted to the central platform 17 via Internet. The physician 195 connects a computer to the central platform 17 to obtain the measured data and makes a diagnosis using the diagnostic interface provided by the central platform 17.

It is to be noted that in the above mentioned architecture, general purpose computers, personal digital assistant, or mobile phones can be used as terminals for connecting to the central platform 17. Also, in the hospital, corresponding measured apparatuses can be deployed for speeding the procedure to see a physician.

In addition to providing an efficient medical providing approach that saves time of patients, the patients 193 can also find a physician 195 who satisfies the requirements of the patient 193 quickly in the preferred embodiment.

It is to be understood that the computerized medical service providing system not only saves patients' time but also helps patients select a physician 195 satisfying requirements of the patients 193 by enhancing functions of the selection interface 192. For example, an expert system is utilized in the selection interface to query symptoms to patients 193 and provide a list of candidate physicians from the database.

Besides, the structure of the central platform 17 is flexible to easily establish web sites of each physician 193 and each medical center 30 and cyber community for particular diseases. Thus, it encourages the physician 195 to provide most advance medical information to win trust of more patients 193. The relationship between patients and physicians are also improved.

In summary, the present invention has at least the following advantages. Firstly, asynchronous medical service is achieved so that vast time of patients is saved, procedure of seeing a physician becomes more efficient, and physicians have more time to improve medical quality and better contents of medical service. Secondly, the patients can select a physician under an efficient interface. Sufficient information and competitive mechanism ensure that the patients have better medical service. Thirdly, it is flexible to organize the relationship between the medical centers, the physicians and patients for better flexibility. Fourthly, the charging mechanism helps the medical centers and the physicians manage charging affairs. Thus, physicians can concentrate more on medical services, and expensive medical equipments can be shared easily between physicians. Fifthly, medical resources, e.g. foreign physicians, may be imported easily by adjusting the logic architecture of the program logic if corresponding regulations are allowed. Sixthly, multiple physicians can cooperate together more efficiently under the flexible medical service providing architecture because it is easy and efficient for physicians to deal with charging affairs and transferring patients' medical history data.

Although the present invention is described using the above embodiments, it does not mean that the scope of the present invention is limited to the above description. Persons skilled in the art can make all kinds of modifications to achieve the same effects. However, these modifications shall not deviate from the spirit of the present invention, and they shall fall within the scope of protection of the present invention. The scope of protection of the present invention shall not be limited to the description of the claims.

What is claimed is:

1. A method for providing medical service, comprising:
receiving a measured data of a patient;
transmitting the measured data to a database and storing the measured data in the database;
assigning a physician based on a selection made by the patient via a selection interface;
providing the measured data to the assigned physician for performing a diagnosis based on the measured data; and
transmitting the result or relevant diagnostic information of the diagnosis to the patient via a transmission interface.

2. The method of claim 1, further comprising:
providing a medical center management unit for registering a plurality of medical centers, wherein each medical center comprises at least one physician to be selected via the selection interface; and
providing a charging interface for allocating a portion of fee charged by the medical center to the assigned physician.

3. The method of claim 2, further comprising:
providing a list of physicians based on a requirement or the measured data transmitted by the patient for the patient's selection.

4. The method of claim 1, further comprising:
coupling the database to an electronic commerce platform for providing service or a commerce transaction; and
allocating a portion of a commission amount to the assigned physician if the patient conducts a commerce transaction or receives service from the electronic commerce platform via the assigned physician's introduction.

5. The method of claim 4, further comprising:
providing a measurement device for measuring vital signs or relevant information of the patient; and
receiving the measured data of the patient via a network.

6. The method of claim 1, further comprising:
registering associated data between a plurality of patients, a plurality of medical centers and a plurality of physicians in the database; and
allocating a portion of a commission amount to a medical center or a physician introducing the patient to the assigned physician.

7. The method of claim 1, further comprising:
transmitting an authenticated prescription to the patient for acquiring medicine according to the authenticated prescription.

8. The method of claim 1, further comprising:
transmitting an authenticated further measurement instruction to the patient for a further measurement and returning the result of the further measurement.

9. The method of claim 1, further comprising:
providing a scheduling interface for scheduling a date for the patient and the assigned physician.

10. The method of claim 1, wherein transmitting the result of the diagnosis to the patient is transmitted by an asynchronous message transmission manner.

11. The method of claim 1, further providing a diagnostic interface for providing the assigned physician auxiliary medical information based on the measured data or medical history of the patient.

12. A system for providing medical service, comprising:
a database for storing a plurality of measured data of a plurality of patients, data of at least one physician and data of at least one associated medical center;
a selection interface coupled to the database for a patient's assignation of a physician;
a diagnostic interface coupled to the database for transmitting associated measured data of the patient to the assigned physician and receiving a diagnosis to the patient made by the assigned physician; and
a transmission interface for transmitting the result of the diagnosis to the patient.

13. The system of claim 12, wherein the diagnosis comprises an authenticated prescription to the patient for acquiring medicine according to the authenticated prescription.

14. The system of claim 12, wherein the transmission interface generates an associated medical information and transmits the associated medical information to the patient together with the diagnosis.

15. The system of claim 12, wherein the diagnostic interface provides the assigned physician auxiliary medical information based on the measured data or medical history of the patient.

16. The system of claim 12, wherein the transmission interface transmits the diagnosis to the patient in an asynchronous message transmission manner.

17. The system of claim 12, further comprising a measurement device for measuring vital signs or relevant information of the patient and retrieving the measured data via a network.

18. The system of claim 12, further comprising a scheduling interface for scheduling a date for the patient and the assigned physician.

19. The system of claim 12, further comprising a charging interface for allocating a fee charged by the medical center to the assigned physician and the associated medical center.

20. A medical measurement apparatus, comprising:
an authentication interface for authenticating an identify of a patient;
a measurement device for measuring vital signs or relevant information of the patient based on a measurement instruction after the authentication;
a transmission interface for transmitting the measured data to a database via a network or a portable storage device such that an assigned physician can perform a diagnosis based on the measured data; and
a controller coupled to the authentication interface, the measurement device and the transmission interface for controlling operation of the medical measurement apparatus.
